(19) United States
(12) Patent Application Publication
(10) Pub. No.: US 2004/0241231 A1
(43) Pub. Date: Dec. 2, 2004

(54) Flat, oral dosage form comprising particles containing active ingredients

(76) Inventor: Frank Becher, Koblenz (DE)

Correspondence Address: BIRCH STEWART KOLASCH & BIRCH, PO BOX 747, FALLS CHURCH, VA, 22040-0747, US

(21) Appl. No.: 10/484,617

(22) PCT Filed: Jul. 11, 2002
(86) PCT No.: PCT/EP02/07717

(30) Foreign Application Priority Data
Jul. 27, 2001 (DE) 101 36 781.3

(51) Int. Cl. A61K 9/26

(52) U.S. Cl. 424/469

(57) ABSTRACT

A form of administration for oral administration of active substances, comprising a carrier matrix and at least one active substance, is characterized in that it has a carrier matrix containing a plurality of particles having open pores or capillary spaces which serve as active substance reservoir and contain at least one active substance.

The invention relates to forms of administration for oral application of active substances, comprising a carrier matrix, and at least one active substance which is contained in particles. More particularly, it relates to flat, oral forms of administration of small thickness.

The manufacture of administration forms is frequently performed in such a manner that the active substance, mixed with suitable carrier substances and auxiliary substances, is incorporated in liquid form in the basic or matrix material of the form of medicament and that then the desired form of administration is produced in further process steps.

This approach is, however, disadvantageous for various reasons, in particular in the manufacture of certain types of administration forms.

If, for example in the manufacture of flat forms of administration to be administered orally (wafer-shaped administration forms, also called “wafers”), the procedure is such that an active substance which is present in liquid form is incorporated into the material of the carrier layer of the wafer, this may have as a consequence that only relatively small amounts of active substance can be incorporated. This is because, on account of the small thickness of such systems, the mechanical properties of the carrier material, especially its coherence and flexibility, would be adversely affected by an active substance load that is too high.

In addition, it has to be taken into consideration that, at the temperatures suitable for the preparation of medicaments, certain active substances are present exclusively as liquids, which means that they can only be processed in liquid form.

The task underlying the invention was therefore to indicate oral forms of administration or forms of medicaments the manufacture of which can start from liquid preparations of active substance, but without the appearance of the abovementioned disadvantages.

This task is solved by forms of administrations according to claim 1 and the processes of manufacture according to claims 19 to 23, as well as by the especially preferred embodiments which are described in the dependent claims.

The invention provides for the carrier matrix of an administration form mentioned in the introductory part of claim 1 to have a plurality of particles containing open-pore or capillary spaces, said particles serving as active substance reservoir.

A particular advantage of this type of administration forms is that the active substance concerned does not have to be distributed uniformly in the carrier matrix or applied thereto in dissolved form, but is instead present in many small reservoir particles. In this way it is possible to considerably reduce the overall amount of active substance as it is not necessary to homogeneously distribute the active substance. It is sufficient for the active substance to be present in many small particles, and only in these, in a sufficient concentration for it to become effective. A further advantage consists in the fact that the firmness of the administration form, respectively of the carrier matrix, is not adversely affected by the active substance present in particle form since the liquid active substance portions are bound in the particles.

1. The particles according to the invention may be particles which have open pores or contain capillary spaces and which have a large interior surface.

2. They may also be such active substance-containing particles as are obtained by the process described in WO 99/17868.

The following initially relates to the first-mentioned form of particles.

These particles serve as active substance reservoir and contain at least one active substance, preferably in liquid form. “Liquid form” is understood to mean that the active substance itself is present in liquid state, or that it is present as a solution, dispersion, suspension, emulsion or as a liquid active substance preparation.

A main advantage of these active substance preparations consists in that the active substance-containing particles are first loaded with liquid active substance or a liquid active substance preparation, in a manner known to those skilled in the art.

This can be done, in particular in an especially preferred embodiment, by placing the particles containing pores or capillary spaces in a vacuum (preferably in the range of approx. 100 to 10-3 mbar, more preferably 10 to 0.01 mbar, and most preferably 1 to 0.1 mbar). This has the special advantage of air, which is present in the capillaries in most cases, being removed; as a consequence, the specific weight of the particles is higher and the particles do no longer float on the surface of the active substance liquid. Still in the vacuum, the particles are virtually washed round with the active substance liquid, which can be achieved, for example, by stirring with high-speed stirring apparatuses, shaking, or in any other suitable way. When normal pressure conditions are subsequently established, the active substance liquid is pressed into the capillaries or pores by the air pressure.

In a further preferred embodiment, the particles are incorporated in the active substance liquid, the latter is then subjected to increased pressure (preferably in the range of from 2 to 300 bar, more preferably 10 to 200 bar, most preferably 10 to 100 bar), so that the active substance liquid is pressed into the air-filled pores. Upon subsequent relief of pressure, the air located in the pores will emerge because the adhesion forces of the liquid are greater.

For impregnation, the processes known to those skilled in the art may be utilized (e.g. pressure chamber impregnation of wood).

In a further preferred embodiment, the particles are heated to high temperatures (preferably in the range of from 40 to 200° C., with particular preference 50 to 150° C.), so that the pressure of the air which is present in the pores is low; these hot particles are then washed round with cold active substance fluid, so that they are able to enter into the cavities. “Cold” means that the temperature is lower than that of the particles.

Furthermore, the loading of the particles can be performed in such a manner that the particles are suspended in and mixed with the liquid active substance, respectively the liquid active substance preparation under normal pressure and at room temperature (approx. 20-30° C.), preferably under stirring.

The above-indicated processes can be combined in a manner known to those skilled in the art, for example by alternate pressure impregnation and vacuum impregnation.

The loaded particles are, if necessary, separated from the excess active substance fluid, for instance by sedimentation or filtration.

Subsequently, the particles, which are loaded with liquid active substance, can be incorporated in solid form, e.g. as a powder, in the carrier matrix of the respective active substance preparation. In this process, no liquid, or only a negligible amount of liquid, is introduced into the carrier matrix mass, so that the structure, consistency, tackiness, elasticity and other properties of the matrix material are not adversely affected. In any case, it is possible in this way to incorporate larger quantities of an active substance present in liquid form into a medicinal preparation than would be the case when employing conventional modes of manufacture. For this reason, in the manufacture of the inventive forms of medicaments it is possible to employ substantially the same methods and apparatuses as are used for the processing of solid medicinal active agents.

In particular, the invention enables the production of flat, thin forms of administration starting from liquid active substance preparations.

It is of particular advantage that different particles can also be loaded with different active substances so that it is readily possible to prepare combined preparations.

It is also advantageous that, in addition, other particles can also be loaded with liquid plasticizers and/or (skin-penetration) enhancers. Alternatively, these substances may also be contained along with the active substance in the same particles.

Plasticizers, respectively enhancers can be selected from the following substances and substance groups:

Saturated or unsaturated fatty acids, hydrocarbons, straight-chain or branched fatty alcohols, dimethyl sulfoxide, propylene glycol, decanol, dodecanol, 2-octyl dodecanol, glycerol, isopropylidene glycerol, transcutol (=diethylene glycol monoethyl ether), DEET (=N,N-diethyl-m-tolueneamide), solketal, ethanol, 1,2-propanediol, or other alcohols, menthol and other ethereal oils or components of ethereal oils, lauric acid diethanolamide, D-alphatocopherol and dexpanthenol; the above list is not complete.

Advantageously, various particle types and sizes can be utilized in order to achieve differentiated release behaviour.

A further advantage consists in the fact that by using liquid-filled active substance particles, it is possible to improve safety in the production of medicines. This is particularly true if there is a risk of the personnel becoming contaminated with active substances, especially with toxic substances.

As porous particles that can be loaded with liquid active substances or active substance solutions, those substances are particularly suitable which are selected from the group comprising activated charcoal particles, particles of porous minerals, especially kieselguhr particles, diatomaceous earth, pumice, lava, bentonite, ceramic or clay particles, silica gel particles, silicon monoxide particles, zeolite, as well as particles of natural or synthetic sponges or of solidified foams. “Porous particles” is understood to also include those particles which have a capillary structure.

A common property of the particles mentioned is that because of their pores or capillaries they have a large internal surface, which is a basic prerequisite for a high active substance load.

Suitable as synthetic sponges or foams are, depending on the intended use, both biodegradable materials (e.g. solidified gelatine foams or collagen foams) and non-degradable materials (e.g. polyurethane foams, microcellular polyester foams or polyether foams).

Also considered are superabsorbers such as polymers which are capable of swelling, as described, for example, in PCT/EP 95/02120.

When selecting the particles, care must be taken that the selected types are suitable, from a pharmacological and toxicological point of view, for the intended mode of application (e.g. oral, transmucosal). Possible interactions with the active substance utilized which are known to those skilled in the art are also to be taken into account and, if possible, to be avoided.

The average particle size of the porous particles is preferably ≦2 mm, more preferably ≦0.5 mm, and even more preferably ≦200 μm, especially ≦50 μm. The particle size may be set, for example, by grinding and/or sieving, but also by growing suitable crystals or by suitable precipitation methods known to those skilled in the art.

The particles used according to the invention are generally finely pored, the average pore or capillary diameter preferably being ≦0.1 mm, more preferably ≦20 μm, and particularly preferably ≦1 μm.

The portion of active substance-containing particles, relative to the carrier matrix, may be varied within a wide range. To achieve a high load of active substance, the active substance-loaded particles may be contained in an administration form at a portion of up to 95%-wt, independently of the carrier matrix selected in a particular case. Therefore, the particle portion preferably amounts to 0.1 to 95%-wt., more preferably 5 to 60%-wt, with particular preference 5 to 25%-wt, each relative to the entire form of administration. Due to the wide range with regard to the particle content as well as with regard to the active substance amount, respectively the active substance concentration, in the individual particles, the inventive forms of administration are able to cover a broad range with regard to dosage.

However, it is to be taken into consideration that an excessively high portion of active substance-loaded particles has an adverse effect on the physical properties of the carrier matrix. The upper limit for this portion can in each individual case be readily determined by experiments.

The loading of these porous or capillary-containing particles with active substance(s) may preferably be performed such that the liquid active substance, an active substance solution, dispersion, suspension or emulsion, or a liquid active substance preparation is mixed with a suitable quantity of particles, whereby the pores or capillary spaces are filled with active substance liquid or solution. Subsequently, the loaded particles can be separated from the excess active substance fluid or solution by methods known to those skilled in the art. Optionally, this may be followed by a drying process to remove any remaining residues of liquid or solvent.

According to a preferred embodiment, an active substance-containing solution is used which contains at least one solid active substance in dissolved form in a suitable solvent. This may also be a saturated active substance solution.

To facilitate entry of the active substance fluid into the interior of the particles upon loading of the particles, it may be necessary to add small amounts of surfactants or emulsifiers.

Apart from the porous particles already mentioned, those active substance-containing particles as can be obtained by the method described in WO 99/17868 and are designated as “concentrated powder form” (CPF) particles are also particularly preferred for making the inventive forms of administration. These are pulverulent, liquid-loaded particles or agglomerates of particles which are formed when an inert gas or gas mixture in a liquid active substance, an active substance-containing solution or suspension or any other liquid active substance preparation is dissolved in said liquid under pressure (preferably ca. 5 to 500 bar, especially preferably from 10 to 250 bar) and the pressure on this solution is subsequently rapidly relieved (by means of a nozzle, for example) while simultaneously adding a pulverulent solid carrier material (carrier particles). The powders obtained in this manner are substantially dry and free-flowing and can contain up to 80%-wt of an active substance liquid. They have the advantage of being able to be processed like solid particles, despite their high liquid content.

The liquid contained is either present in the capillary spaces of the aggregated carrier particles, and/or in the pores of the carrier particles if open-pore carrier particles are used.

As pulverulent carrier substances, respectively carrier particles, one may, for instance, use starch types (such as maize, potato, wheat starch), silicic acid, respectively silicon dioxide, celluloses (e.g. microcrystalline celluloses, cellulose derivatives such as carboxymethyl celluloses, cellulose fibres); moreover it is also possible to use as particles porous particles of the kind mentioned at the outset, such as activated charcoal, zeolite, silicic acid, or polymers capable of swelling (especially so-called superabsorber polymers). “Polymers capable of swelling” is understood to preferably mean water-swellable polymers, e.g. polyvinyl alcohols with a high degree of hydrolysis, or high-molecular hydroxypropyl methyl cellulose. The pulverulent carrier particles preferably have a particle size of less than 100 μm.

Further auxiliary substances considered for the manufacture of the pulverulent, liquid-filled particles are: common salt, sugar, dextrin, proteins, titanium dioxide, fats, polyglycols, magnesium stearate, highly dispersed silicon dioxide, glutamate, calcium, kaolin, polylactic acid, fats, waxes, thickeners.

To facilitate a subsequent resuspension of the liquid-filled particles it is also possible to add emulsifiers, such as phospholipids, especially lecithin, or partial glycerides during manufacture.

Inert gases considered are first of all carbon dioxide, gaseous hydrocarbons (e.g. methane, ethane, propane, butane), ether, nitrogen, dinitrogen monoxide, ammonia or inert gases.

The particles loaded according to the described process, too, will, if required, be separated from the excess active substance fluid, for instance by sedimentation or filtration.

Further processing and use of these particles, especially the manufacture of the inventive administration forms, may be performed in a fashion corresponding to that described above with respect to the first-mentioned form of the particles.

The following remarks apply to all of the various types of particles described:

According to a preferred embodiment, it is further provided for the particles, or at least a portion thereof, to be provided with a coating of fat- and/or water-soluble substances. In this way it is, for instance, possible to achieve a control of the active substance release, in particular a control of the active substance release rate, or to improve the water-wettability. Materials considered for such coatings are, inter alia: film formers (e.g. polyacrylates, polymethacrylates), polyethylene glycols, vegetable or animal oils, liquid paraffin, polyvinyl pyrrolidone, cellulose derivatives.

To prevent that the active substance contained in the particles after oral administration of a medicament form are already released in the stomach, it is possible to provide the particles, after loading the same with active substance, with an enteric, small intestine-soluble coating. This is particularly advisable in the case of active substances which irritate the gastric mucosa or would be disintegrated under the influence of the gastric juice. Suitable as enteric, small intestine-soluble coatings are first of all cellulose derivatives such as cellulose acetate phthalate, cellulose acetate succinate and hydroxyethyl cellulose, methacrylic acid/ethyl acrylate copolymers as well as certain copolymerisates of acrylic acid, methacrylic acid, and esters thereof which are known to those skilled in the art.

The present invention relates, in particular, to flat forms of administration to be administered orally, e.g. wafer-shaped forms (“wafers”); this embodiment is preferred most. The thickness of these flat forms of medicament preferably lies in the range of 0.1 to 5 mm, with particular preference in the range of 0.1 to 1 mm. In such flat and thin administration forms, the advantages arising from the invention are particularly useful since the invention enables the production of wafers having a high portion of liquid active substance while leaving the physical properties of the carrier matrix substantially unaffected. The liquid active substance portion may be up to 60%-wt, and depending on the thickness and the type of carrier matrix even up to 80%-wt.

In this connection, materials considered for the carrier matrix are first of all water-soluble and, in particular, film-forming polymers, or mixtures of such polymers, these may be synthetic or partially synthetic polymers, or biopolymers of natural origin. Particularly suitable are polymers which are selected preferably from the group comprising cellulose derivatives, polyvinyl alcohol, polyacrylates and polyvinyl pyrrolidone. Particularly preferred cellulose derivatives are hydroxypropyl methyl cellulose, carboxymethyl cellulose, hydroxypropyl cellulose and methyl cellulose. Also preferred are water-soluble polysaccharides which are of vegetable or microbial origin, especially pullulan, xanthan, alginate, starch, dextranes and pectins. Furthermore, proteins, preferably gelatine or other gel-forming proteins are also suitable.

The inventive principle may, however, also be applied in the manufacture of other flat, oral forms of administration such as tablets, coated tablets, chewable tablets, sucking tablets, lozenges or sublingual tablets. The basic substances, binding agents and other auxiliary substances suitable for the manufacture of these medicament forms and, in these cases, serving for the production of the carrier matrix, are basically known to those skilled in the art (e.g. starch, starch hydrolysates, cellulose, cellulose derivatives, sugars, gelatine, synthetic polymers such as polyethylene glycols, polyvinyl alcohols, polyvinyl pyrrolidones, poly(meth)acrylates).

Apart from administration forms wherein the active substance-containing porous particles are embedded in a carrier matrix, the invention also comprises such forms of medicaments where the active substance-loaded particles are applied to at least one surface of the medicament form. This can be done by providing the surface of the carrier matrix with pressure-sensitive adhesive properties.

A further preferred embodiment of the invention relates to oral administration forms which have mucoadhesive properties and enable transmucosal administration of active substances. These can be, in particular, medicament forms for buccal or sublingual application. In this case it is provided that either the carrier matrix itself has mucoadhesive properties or that at least one surface of the administration form will be equipped with mucoadhesive properties, e.g. a mucoadhesive coating. Mucoadhesive properties can be achieved, for example, by using or adding substances such as starch, carboxymethyl cellulose sodium, carboxymethyl cellulose, hydroxypropyl cellulose, hydroxypropyl cellulose, polyacrylic acid, polyvinyl pyrrolidone, polyethylene oxide polymers, ethyl or propyl cellulose, alginates, pectins or natural rubbers.

Furthermore, the oral preparations according to the invention, especially flat medical forms such as “wafers”, may be formulated as preparations which are disintegratable in aqueous media (e.g. in saliva). This can be achieved by using water-soluble base substances for making the carrier matrix and adding disintegration-promoting auxiliaries, e.g. starch, cross-linked polyvinyl pyrrolidones.

The carrier matrix, in which the active substance-containing particles are embedded, may optionally contain auxiliary substances apart from the matrix-forming base materials. Considered for this purpose are filling agents (e.g. SiO₂); thickening agents (e.g. alginates, pectin); colourants (e.g. quinoline yellow or TiO₂); disintegrants, especially disintegrants which draw water into the matrix and explode the matrix from within (e.g. aerosil); emulsifiers (e.g. polyethoxylated sorbitan fatty acid esters such as TWEEN® or polyethoxylated fatty alcohols such as BRIJ®; plasticizers (e.g. polyethylene glycol, glycerol); sweeteners (e.g. aspartame, saccharine); preservative agents (e.g. sorbic acid and its salts), and flavouring agents.

If a medicament is to be produced which is to be capable of swelling in aqueous media, there may be added to the carrier matrix, for example, hydrophilic swelling agents such as polyhydroxyalkyl methacrylates having a molecular weight of 5,000 to 5,000,000, mixtures of agar and carboxymethyl cellulose, agents capable of swelling and consisting of methyl cellulose in admixture with loosely cross-linked agar, tragacanth, gelatine or ion exchanger resins which are capable of swelling.

“Active substances/agents” are understood to mean any medicinal agents used in field of human or veterinary medicine, including vitamins, enzymes and hormones, as well as active substances for cosmetic treatments, and flavouring agents or aromatics. More particularly, the invention relates to medicinal active substances which can be absorbed by the oral mucosa or which can be taken up via the gastrointestinal tract. Especially preferred are active agents which are present in liquid state; the invention is furthermore applicable to a plurality of further active substances which can be brought into a liquid form, for instance as a solution, dispersion, suspension or emulsion.

The release of active substance(s) contained in the inventive forms of administration can be achieved in different ways. After oral administration or application to a mucosal surface, the active substance is able to diffuse out of the particles and be subsequently absorbed. If the administration form is constituted so as to be disintegratable, the particles can initially be released as such and subsequently the active agent contained in the particles can be released. If the particles are made of biodegradable material, the release may be influenced or accelerated by degradation of the particle material. In this way the present invention opens numerous possibilities of controlling the delivery of active substance.

The invention further comprises processes for the manufacture of oral forms of medicaments which start from liquid active agents, active agent solutions or active agent preparations.

The inventive forms of administration may preferably be obtained by initially providing a carrier matrix material as described above—which is suitable for the desired form of medicament, preferably in liquid or semi-solid form (e.g. as a solution or melt), or as a gel.

Then, a liquid active substance, an active substance solution or a liquid active substance preparation is provided. If the active substance itself is not present in liquid form, it is dissolved, dispersed or suspended in a pharmaceutically acceptable solvent or solvent mixture suitable for the active agent. The liquid active agent preparations may furthermore also contain active substance combinations. In a next process step, the liquid active substance, respectively the active substance solution, is mixed with particles having open pores or having capillary spaces (as described above), whereby the pores or capillary spaces will be filled with active substance liquid or active substance solution. This process can be assisted by addition of surfactants or emulsifiers.

After separating the particles from the excess active substance liquid or solution, optionally followed by a drying step, the particles, loaded with active substance liquid, are introduced into the carrier material mentioned in the first step and are incorporated therein and mixed therewith, so that the particles are homogeneously distributed in the carrier matrix. If necessary, wetting agents (surfactants, e.g. SDS), emulsifiers (e.g. lecithin) etc. can be admixed in order to improve the dispersion of the particles in the carrier matrix material.

Finally, depending on the type of medicament form to be made, it is possible to add and incorporate auxiliary agents (as mentioned above), and to carry through a further drying to achieve the desired consistency of the carrier matrix by solvent withdrawal.

The further processing of the medicament forms may be performed using conventional methods, e.g. pressing, punching or coating.

Flat oral administration forms, e.g. wafer-shaped administration forms (“wafers”) can be obtained by casting or coating the still-liquid carrier matrix mass with the porous particles contained therein, in a thin layer onto a suitable film-shaped support (e.g. polyester film, PET). After drying thereof, individual wafers can be produced by cutting or punching.

The above-described process may be modified in various ways. The porous particles loaded with active substance may, for example, be provided with a coating prior to embedding, which coating prevents the diffusion of the active substance into the matrix (or into the solvent) as long as the matrix has not yet dried or solidified. Likewise, the particles may be provided with a fat-soluble and/or water-soluble coating or with an enteric coating prior to embedding, as mentioned hereinabove.

In a further, preferred manufacturing process of forms of medicaments for transdermal, transmucosal or epicutaneous administration forms, it is provided, as a modification of the above-described processes, that the production of the active substance-loaded particles is carried out in accordance with the process described in WO 99/17868, as described above (“concentrated powder form” (CPF) particles).

This active substance-containing powder is then embedded in the carrier material, which his present in liquid or semi-solid form; further processing is carried out as described above.

Furthermore, this process of manufacture, too, can be modified in different ways, for example by applying coatings or covers to the particles prior to the embedding step.

Thus, the present invention advantageously enables the production of oral forms of medicaments, especially of flat medicament forms, which can have a high content of an active substance present in liquid form.

1. Flat, wafer-shaped form of administration for oral administration of active substances, comprising a carrier matrix and at least one active substance, characterized in that said form of administration has a carrier matrix containing a plurality of particles having open pores or capillary spaces which serve as active substance reservoir and contain at least one active substance.

2. Form of administration according to claim 1, characterized in that the particles are pulverulent particles or particle agglomerates loaded with liquid which were manufactured by dissolving, under pressure, an inert gas in an active substance-containing solution or suspension and subsequently releasing the pressure on the solution or suspension while simultaneously admixing of a pulverulent, solid carrier material.

3. Form of administration for oral administration of active substances, comprising a carrier matrix and at least one active substance, characterized in that said form of administration has a carrier matrix containing a plurality of particles having open pores or capillary spaces which serve as active substance reservoir and contain at least one active substance, said particles being pulverulent particles or particle agglomerates loaded with liquid which were manufactured by dissolving an inert gas under pressure in an active substance-containing solution or suspension and subsequently releasing the pressure on the solution or suspension while simultaneously admixing of a pulverulent, solid carrier material.

4. Form of administration according to claim 2 or 3 characterized in that the particles contain swellable, liquid-absorbing polymers, preferably superabsorbing polymers, as carrier material.

5. Form of administration according to an one of claims 1 to 4, characterized in that the porous particles are selected from the group comprising activated charcoal particles, particles of porous minerals, especially kieselguhr particles, ceramic or clay particles, silica gel particles, zeolite particles, as well as particles of natural or synthetic sponges or of solidified foams.

6. Form of administration according to claim 1 or 5, characterized in that the average particle size of the particles is ≦2 mm, preferably ≦0.5, more preferably ≦200 μm.

7. Form of administration according to any one of claims 1 to 6 characterized in that the particles having pores or capillary spaces are finely pored, with an average pore or capillary diameter of ≦0.1 mm, preferably ≦20 μm, especially of 1 μm.

8. Form of administration according to any one of the preceding claims, characterized in that the portion of the particles, relative to the carrier matrix, amounts to 0.1 to 95%-wt, preferably 5 to 60%-wt.

9. Form of administration according to one or more of the preceding claims, characterized in that the particles contain the active substance(s) in liquid form or contain an active substance-containing solution which contains at least one solid active substance in dissolved form in a suitable solvent, preferably a saturated active substance solution.

10. Form of administration according to one or more of the preceding claims, characterized in that the particles are applied to at least one surface of the carrier matrix.

11. Form of administration according to one or more of the preceding claims, characterized in that at least a partial amount of the particles is provided with a coating of fat-soluble and/or water-soluble substances, preferably an enteric coating.

12. Forms of administration according to one or more of the preceding claims, characterized in that they contain particles loaded with different active substances.

13. Forms of administration according to one or more of the preceding claims characterized in that different particle types and particle sizes are used.

14. Forms of administration according to one or more of the preceding claims, characterized in that they contain particles loaded with liquid plasticizers and/or permeation enhancers.

15. Forms of administration according to one or more of the preceding claims, characterized in that they contain particles which are water-soluble or biodegradable.

16. Form of administration according to any one of the preceding claims characterized in that it has mucoadhesive properties and is suitable for buccal or sublingual application.

17. Form of administration according to any one of the preceding claims, characterized in that it is substantially flat, its thickness preferably amounting to 0.1 to 5 mm.

18. Forms of administration according to any one of claims 2 to 17, characterized in that they are formulated as a tablet, coated tablet, chewable tablet, sucking tablet, lozenge or sublingual tablet.

19. Forms of administration according to any one of claims 2 to 17, characterized in that they are formulated as wafer-shaped forms of medicaments (“wafers”).

20. Process for the production of a flat, wafer-shaped pharmaceutical preparation for oral administration of active substances, characterized by the following steps:
a) Providing the carrier matrix material in liquid or semi-solid form, or as a gel;
b) providing a liquid active substance or an active substance solution, or a liquid active substance preparation;
c) mixing the liquid active substance, respectively the active substance solution, with particles having open pores or having capillary spaces, thereby filling the pore or capillary spaces with active substance liquid or active substance solution;
d) separating the particles from the excess active substance fluid or solution;
e) introducing the active substance-containing particles into the carrier material mentioned in the first step, and mixing;
f) if necessary, adjusting the desired consistency of the carrier material by solvent withdrawal, especially by drying, or cooling.

21. The process according to claim 20, characterized in that the loading of the particles described in step (c) is carried out at increased pressure or under vacuum conditions, preferably by pressure impregnation in a pressurized chamber.

22. The process according to claim 20, characterized in that the loading of the particles described in step (c) is performed in such a manner that the particle-containing active substance fluid is subjected to increased pressure and the pressure is subsequently relieved.

23. The process according to claim 20, characterized in that the loading of the particles described in step (c) is performed in such a manner that the particles are heated and subsequently mixed with active substance liquid.

24. Process for the production of a pharmaceutical preparation for oral administration of active substances, characterized by the following steps:
a) Providing the carrier matrix material in liquid or semi-solid form, or as a gel;
b) providing a liquid active substance or an active substance solution, or a liquid active substance preparation, in a pressurized vessel;
c) dissolving an inert gas in the liquid active substance or active substance solution, under increased pressure;
d) relieving the pressure on the pressurized solution from step (c), while simultaneously admixing a solid, pulverulent carrier substance, whereby a liquid-loaded active substance-containing powder is formed;
e) introducing the active substance-containing powder from step (d) into the carrier material mentioned in the first step, and mixing;
f) if necessary, adjusting the desired consistency of the carrier material by solvent withdrawal, especially by drying, or cooling.
